(19) United States
(12) Patent Application Publication
(10) Pub. No.: US 2025/0288614 A1
(43) Pub. Date: Sep. 18, 2025

(54) COMPOSITIONS AND METHODS FOR TREATING ENDOMETRIAL TISSUE

(71) Applicant: MAYO FOUNDATION FOR MEDICAL EDUCATION AND RESEARCH, Rochester, MN (US)

(72) Inventors: Timothy Peterson, Rochester, MN (US); Atta Behfar, Rochester, MN (US); Zaraq Khan, Rochester, MN (US)

(21) Appl. No.: 18/702,598

(22) PCT Filed: Oct. 19, 2022
(86) PCT No.: PCT/US2022/047119

§ 371 Date: Apr. 18, 2024

Related U.S. Application Data
(60) Provisional application No. 63/257,454, filed on Oct. 19, 2021.

(51) Int. Cl. A61K 35/19; A61P 15/02

(52) U.S. Cl. CPC A61K 35/19; A61P 15/02

(57) ABSTRACT

A method for treating a subject having. or at risk of having. damaged endometrial tissue generally includes administering to the subject an amount of a PEP preparation effective to treat the subject's endometrial tissue. The PEP preparation may be administered therapeutically or prophylactically. In another aspect. a method of promoting proliferation of endometrial cells generally includes contacting the endometrial cells with an amount of a PEP preparation effective to promote proliferation of the endometrial cells. In another aspect. a method of promoting wound closure by endometrial cells generally includes administering to endometrial cells into which a wound has been introduced an amount of a PEP preparation effective to promote closure of the wound.

### CROSS-REFERENCE TO RELATED APPLICATIONS

This application claims the benefit of U.S. Provisional Application Ser. No. 63/257,454, filed Oct. 19, 2021, which is incorporated by reference herein in its entirety.

### SUMMARY

This disclosure describes, in one aspect, a method for treating endometrial tissue in a subject. Generally, the method includes administering to the subject an amount of a purified exosome product (PEP) preparation effective to treat the subject's endometrial tissue.

In one or more embodiments, the PEP preparation is administered therapeutically to a subject having damaged endometrial tissue. In some of the embodiments, the subject may have Asherman's Syndrome. In other embodiments, the subject may have undergone uterine surgery.

In one or more embodiments, the PEP preparation is administered prophylactically to a subject at risk of developing damaged endometrial tissue. In some of these embodiments, the PEP preparation is administered to the subject prior to uterine surgery.

In one or more embodiments, the PEP preparation is administered preparationally to a subject to prepare the endometrium for fertilization.

In another aspect, this disclosure describes a method of promoting proliferation of endometrial cells. Generally, the method includes contacting the endometrial cells with an amount of a PEP preparation effective to promote proliferation of the endometrial cells.

In another aspect, this disclosure describes a method of promoting wound closure by endometrial cells. Generally, the method includes administering to endometrial cells into which a wound has been introduced an amount of a PEP preparation effective to promote closure of the wound.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Confocal microscope images of endometrial adenocarcinoma cells. PEP was labeled with DiR lipophilic dye prior to being added to cells in culture. After co-incubation, cells were fixed and stained with DAPI, phalloidin, or anti-CD63 fluorescent antibody. Confocal fluorescence microscopy was then performed to obtain representative images of the DAPI (blue, far left), phalloidin (green, second from left), CD63 (red, middle), and DiR-labeled PEP (violet, second from right) and used to generate a merged image (far right). Scale bar=20 μm.

FIG. 2. DiR and CD63 fluorescent signals were quantified using ImageJ and compared between the PEP-treated cells and control cells using a T-test. (A) HEC-1A cells staining with DiR. (B) HEC-1A cells stained with anti CD63 antibodies. There was a statistically higher amount of both CD63 and DiR found in the Pep-treated cells compared to the control cells.

FIG. 3. Confocal microscope images of human endometrial stromal cells (HESCs). PEP was labeled with DiR lipophilic dye prior to being added to cells in culture. After co-incubation, cells were fixed and stained with DAPI, phalloidin, or anti-CD63 fluorescent antibody. Confocal fluorescence microscopy was then performed to obtain representative images of the DAPI (blue, far left), phalloidin (green, second from left), CD63 (red, middle), and DiR-labeled PEP (violet, second from right) and used to generate a merged image (far right). Scale bar=20 μm.

FIG. 4. DiR and CD63 fluorescent signals were quantified using Image J and compared between the PEP-treated cells and control cells using a T-test. (A) HESC stained with DiR. (B) HESC stained with anti-CD63 antibody. There was a statistically higher amount of both CD63 and DiR found in the PEP-treated cells compared to the control cells.

FIG. 5. Representative images at three time points showing increased cell proliferation in HEC-1A cells treated with PEP exosomes versus untreated HEC-1A cells.

FIG. 6. Line graph showing HEC-1A proliferation over the course of 48 hours after treatment with various doses of PEP exosomes compared to untreated HEC-1A cells.

FIG. 7. Representative images at three time points showing increased cell proliferation in HESCs treated with PEP exosomes versus untreated HESCs.

FIG. 8. Line graph showing HESC proliferation over the course of 48 hours after treatment with various doses of PEP exosomes compared to untreated HESCs.

FIG. 9. Representative images of HEC-1A cells at time of scratch wound and treatment with serum free media (Control) or PEP exosomes (1×10¹² PEP exosomes/mL), 12 hours after scratch wound and treatment, and 36 hours after scratch wound and treatment.

FIG. 10. Line graph showing wound healing by HEC-1A cells treated with various doses of PEP exosomes compared to untreated HEC-1A cells.

FIG. 11. Representative images of HESCs at time of scratch wound and treatment with serum-free media (Control) or PEP exosomes (1×10¹² PEP exosomes/ml), 12 hours after scratch wound and treatment, and 36 hours after scratch wound and treatment.

FIG. 12. Line graph showing wound healing by HESCs treated with various doses of PEP exosomes compared to untreated HESCs.

### DETAILED DESCRIPTION OF ILLUSTRATIVE EMBODIMENTS

This disclosure describes compositions and methods for treating tissues of the uterus and/or cervix. The compositions include a Purified Exosome Product (PEP) derived from platelet exosomes through a cryodesiccation process.

The human endometrium consists of a single layer of columnar epithelium overlying a layer of connective tissue or stroma and can be divided into an upper ⅔ functionalis layer and lower ⅓ basalis layer. During a woman's reproductive life, the functionalis layer undergoes monthly growth, shedding, and repair. The endometrium has a remarkable regenerative capacity that is initiated in the lower basalis layer to build a new functionalis layer following menstrual loss.

In Asherman's Syndrome, endometrial damage causes disruption of the endometrial regeneration process leading to a scarred cavity and the loss of a functional endometrium. Patients may develop sequelae including, but not limited to, menstrual changes, cyclic pelvic pain, and infertility. Although the etiology is not fully understood, Asherman's syndrome most commonly results from a traumatic denudation of the basalis layer to raw myometrium in a hypoestrogenic state. The goal of Asherman's Syndrome treatment is to restore an anatomically and physiologically functional cavity. The gold standard for restoration of the anatomical cavity is hysteroscopic lysis of adhesions. However, there is no clear consensus on the best way to regenerate the functional endometrium and prevent reformation of adhesions after hysteroscopic lysis. Regenerative technologies have received increasing attention as both treatments and preventative measures for Asherman's syndrome.

This disclosure describes compositions that include PEP and therapeutic methods of using PEP compositions to treat the stratum basalis and/or functionalis of the endometrium. Compositions that include PEP exosomes induce endometrial regeneration in two different human endometrial cell lines: HEC-1A cells and human endometrial stromal cells (HESCs). HEC-1A is a stage IA endometrial cancer cell line. HESC is an immortalized stromal cell line with a normal karyotype that responds to hormonal stimulation. Data presented in this disclosure show that both endometrial cells lines take up PEP exosomes, PEP composition induce cell proliferation in both cell lines, and PEP compositions increase wound closure in both cell lines. These results suggest that PEP compositions can be an effective therapeutic and/or prophylactic therapy for repairing damaged endometrial tissue and/or reducing damage to endometrial tissues.

PEP is characterized and methods for preparing PEP are described in International Patent Application NO. PCT/US2018/065627 (published as International Publication No. WO 2019/118817), which is incorporated by reference herein in its entirety. Briefly, PEP is a purified exosome product prepared using a cryodesiccation step that produces a product having a structure that is distinct from exosomes prepared using conventional methods. For example, PEP typically has a spherical or spheroidal structure rather than a crystalline structure. The spherical or spheroid exosome structures generally have a diameter of no more than 300 nm. Typically, a PEP preparation contains spherical or spheroid exosome structures that have a relatively narrow size distribution. In some preparations, PEP includes spherical or spheroidal exosome structures with a mean diameter of about 110 nm±90 nm, with most of the exosome structures having a mean diameter of 110 nm±50 nm such as, for example, 110 nm±30 nm.

An unmodified PEP preparation—i.e., a PEP preparation whose character is unchanged by sorting or segregating populations of exosomes in the preparation—naturally includes a mixture of CD63⁺ and CD63⁻ exosomes. Because CD63⁻ exosomes can inhibit unrestrained cell growth, an unmodified PEP preparation that naturally includes CD63⁺ and CD63⁻ exosomes can both stimulate cell growth for wound repair and/or tissue regeneration and limit unrestrained cell growth.

Further, by sorting CD63⁺ exosomes, one can control the ratio of CD63⁺ exosomes to CD63⁻ exosomes in a PEP product by removing CD63⁺ exosomes from the naturally-isolated PEP preparation, then adding back a desired amount of CD63⁺ exosomes. In one or more embodiments, a PEP preparation can have only CD63⁻ exosomes.

In one or more embodiments, a PEP preparation can have both CD63+ exosomes and CD63⁻ exosomes. The ratio of CD63⁺ exosomes to CD63⁻ exosomes can vary depending, at least in part, on the quantity of cell growth desired in a particular application. For example, a CD63⁺/CD63⁻ exosome ratio provides desired cell growth induced by the CD63⁺ exosomes and inhibition of cell growth provided by the CD63⁻ exosomes achieved via cell-contact inhibition. In certain scenarios, such as in tissues where non-adherent cells exist (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁺ exosome ratio to avoid uncontrolled cell growth. Conversely, if there is a desire to expand out a clonal population of cells, such as in allogeneic cell-based therapy or immunotherapy, one can increase the ratio of CD63⁺ exosomes to ensure that a large population of cells can be derived from a very small source.

Thus, in various embodiments, the ratio of CD63⁺ exosomes to CD63⁻ exosomes in a PEP preparation may be 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, or 30:1. In certain embodiments, the PEP product is formulated to contain a 9:1 ratio of CD63⁺ exosomes to CD63⁻ exosomes.

### PEP Uptake by Cell Lines

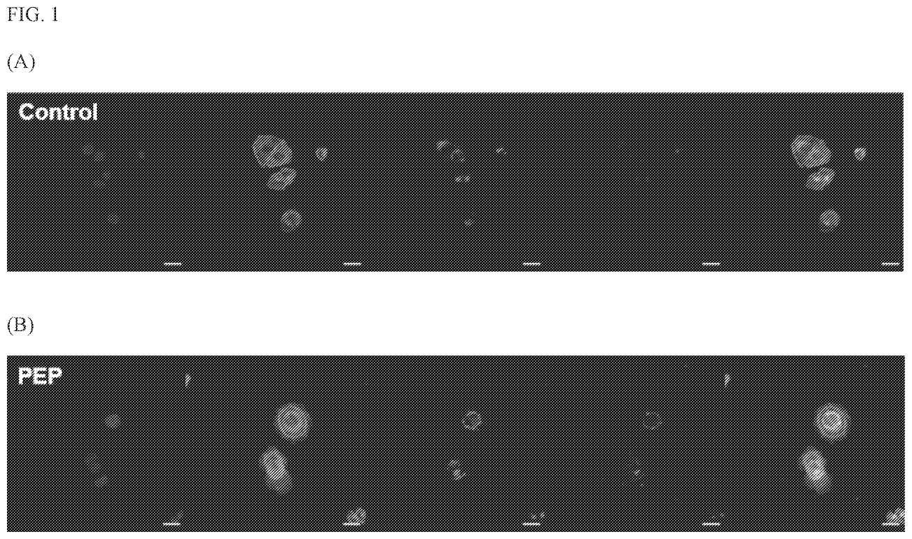

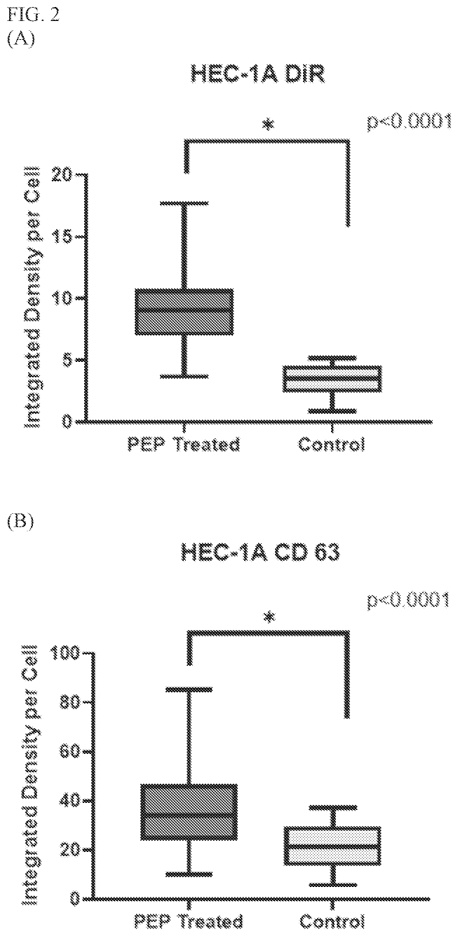

FIG. 1 shows confocal microscope images of endometrial adenocarcinoma cells incubated with DiR-labeled PEP exosomes. After co-incubation, cells were fixed and stained with DAPI, phalloidin, or anti-CD63 fluorescent antibodies. Control cells stained positive for CD63 since HEC-1A is a human cell line and the cells therefore contains CD63+ exosomes as part of normal cell functions. For this reason, DiR staining was used to distinguish endogenous exosomes from PEP exosomes, which can be seen to have a distinct perinuclear localization. Exosomes that are recognized by cells get readily taken up and sent to a perinuclear region of the cell. DiR and CD63 staining were quantified using Image J. There was a statistically higher amount of both CD63 and DiR found in the PEP-treated cells compared to the control cells (FIG. 2).

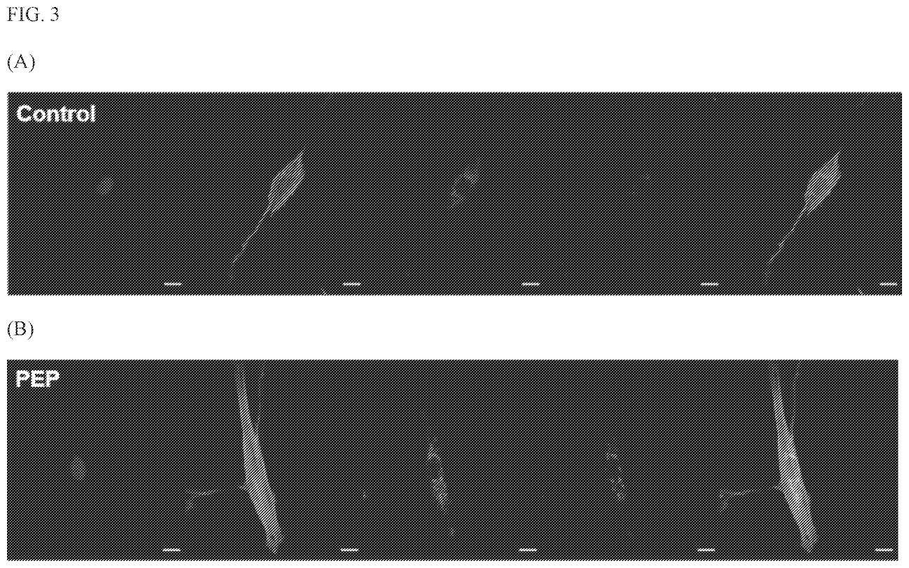

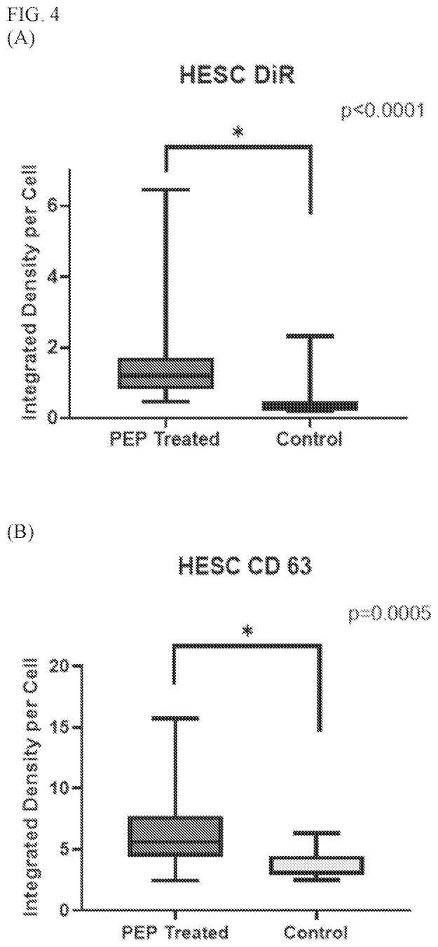

FIG. 3 shows confocal microscope images of human endometrial stromal cells (HESCs) incubated with DiR-labeled PEP exosomes. After co-incubation, cells were fixed and stained with DAPI, phalloidin, or anti-CD63 fluorescent antibodies. Control cells once again stained positive for CD63. DiR staining once again shows perinuclear localization of PEP exosomes. DiR and CD63 staining were quantified using Image J and the Mann-Whitney U test due to nonparametric distribution. There was a statistically higher amount of both CD63 and DiR found in the PEP-treated cells compared to the control cells (FIG. 4).

### Cell Proliferation

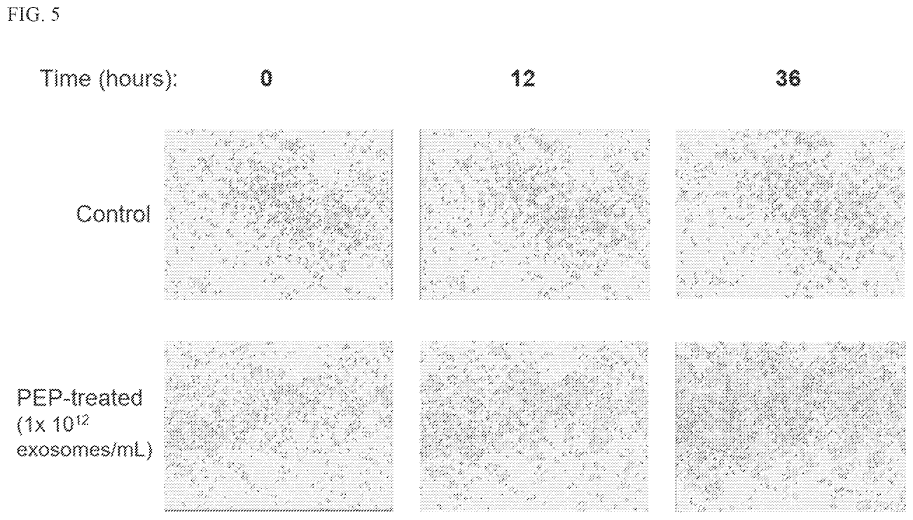

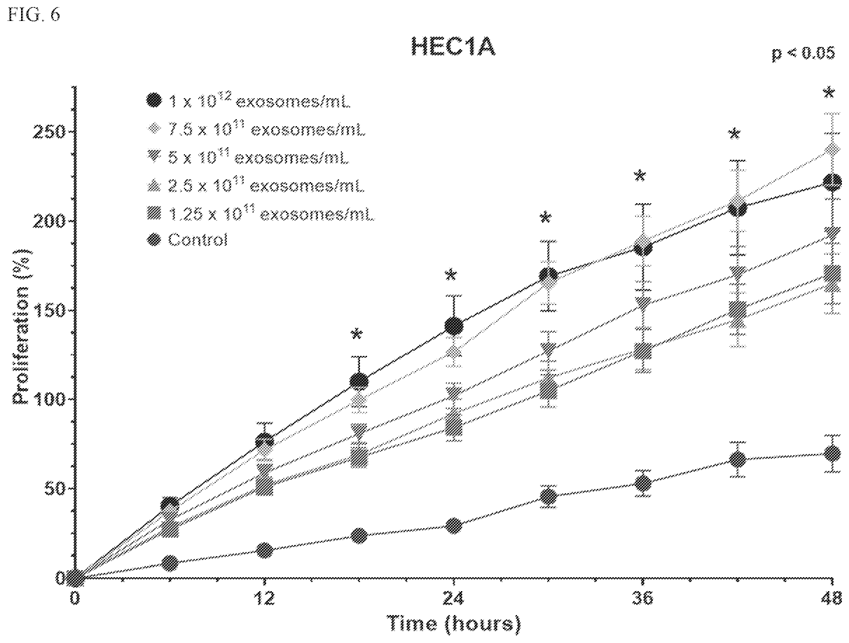

FIG. 5 shows microscope images showing that HEC-1A cells treated with 10¹² PEP exosomes/ml showed increased cell proliferation compared to untreated HEC-1A cells. Images were taken 12 hours after treatment and 36 hours after treatment. Increased cell proliferation and coverage of the tissue culture well were clearly visible at both time points. FIG. 6 shows that by 12 hours post-treatment, cell proliferation was increased in PEP-treated HEC-1A cells compared to the untreated control HEC-1A cells for all PEP dosages tested, 1.25×10¹¹ PEP exosomes/ml up to 10¹² PEP exosomes/ml.

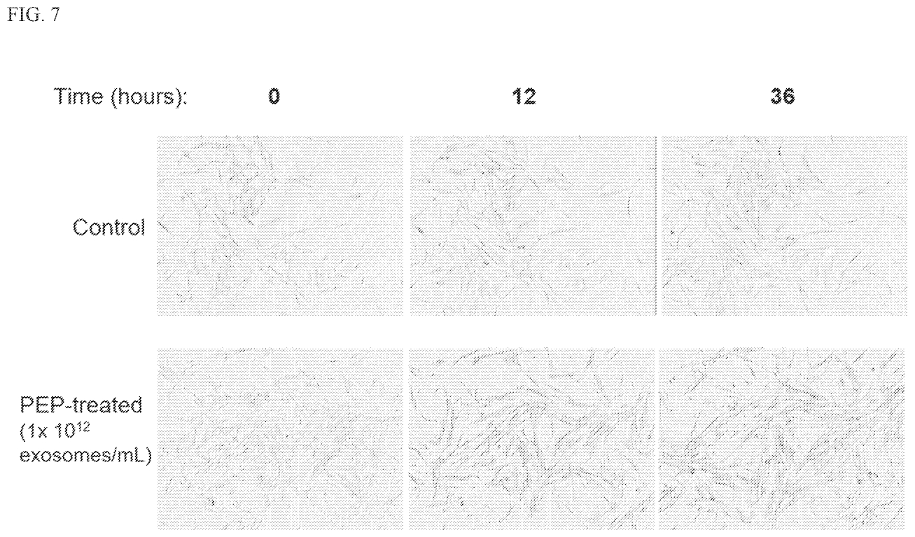

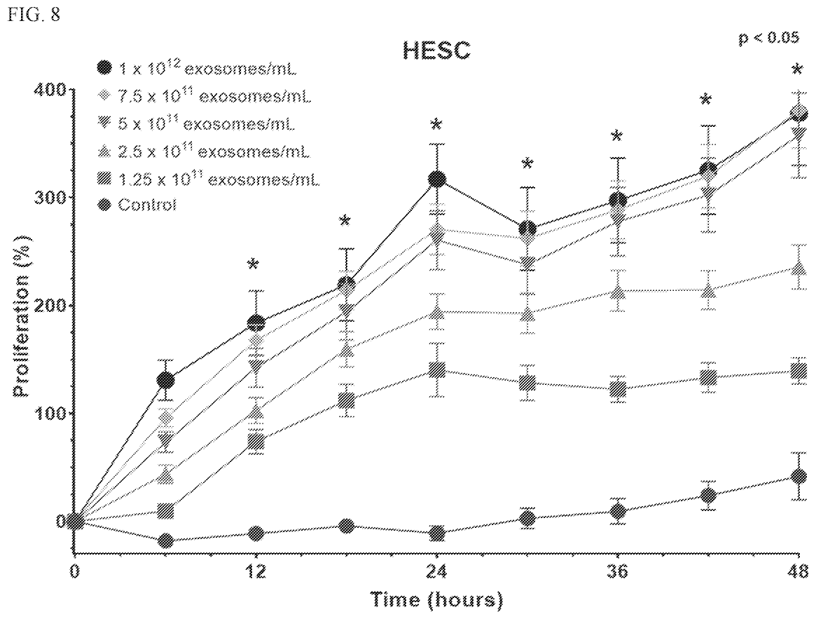

FIG. 7 shows microscope images showing that HESCs treated with 10¹² PEP exosomes/ml showed increased cell proliferation compared to untreated HESCs. Increased cell proliferation is clearly visible at 12 hours and 36 hours after treatment, as evidenced by increased cell density within the tissue culture well. FIG. 8 shows that by 12 hours post-treatment, cell proliferation was increased in PEP-treated HESCs compared to the untreated control HESCs for all PEP dosages tested, 1.25×10¹¹ PEP exosomes/ml up to 10¹² PEP exosomes/ml.

### PEP Effects on Wound Healing

The scratch assay was used to model wound healing in vitro. Briefly, cells were plated in a 96-well tissue culture plate and allowed to proliferate until forming a confluent monolayer. Then, a uniform scratch was made in the center of each well to simulate a wound. Wells were then treated with either serum free media (control) or serum free media supplemented with PEP exosomes.

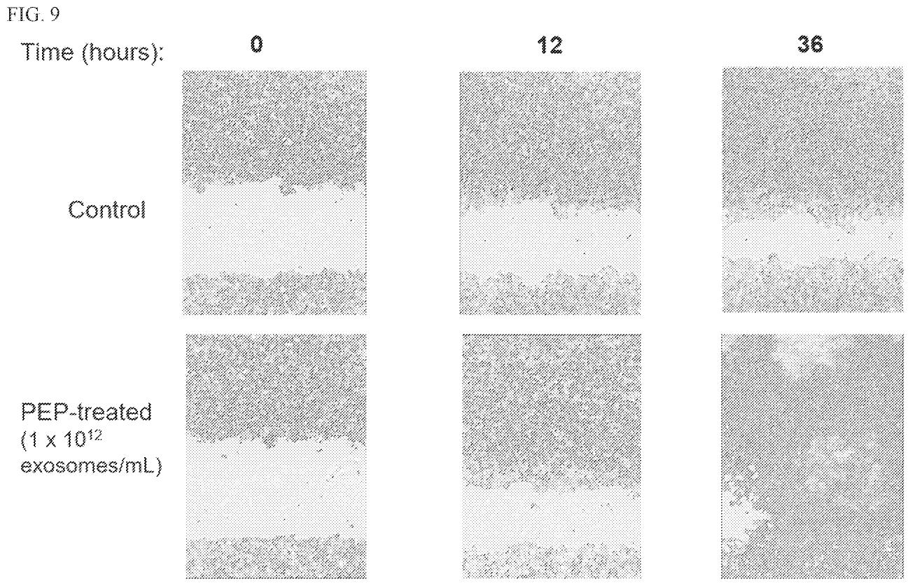

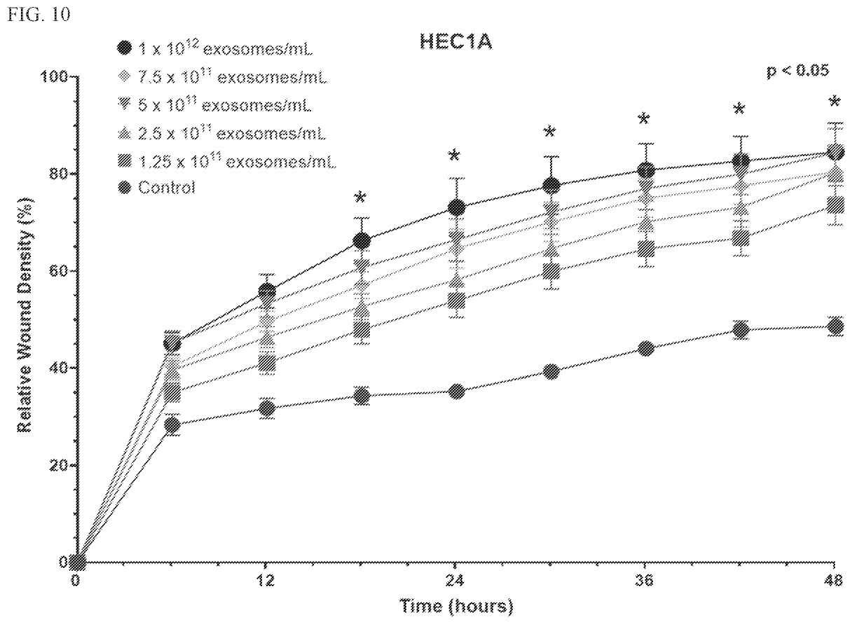

FIG. 9 shows a representative image of wound closure in HEC-1A cells at 12 hours and 36 hours after treatment. The wound closure in the PEP-treated HEC-1A cells was noticeably greater than the untreated HEC-1A cells. FIG. 10 shows the wound healing abilities of different concentrations of PEP exosomes. Eighteen hours after treatment, all concentrations of PEP exosomes induced greater wound closure in HEC-1A cells than was induced in the untreated HEC-1A cells.

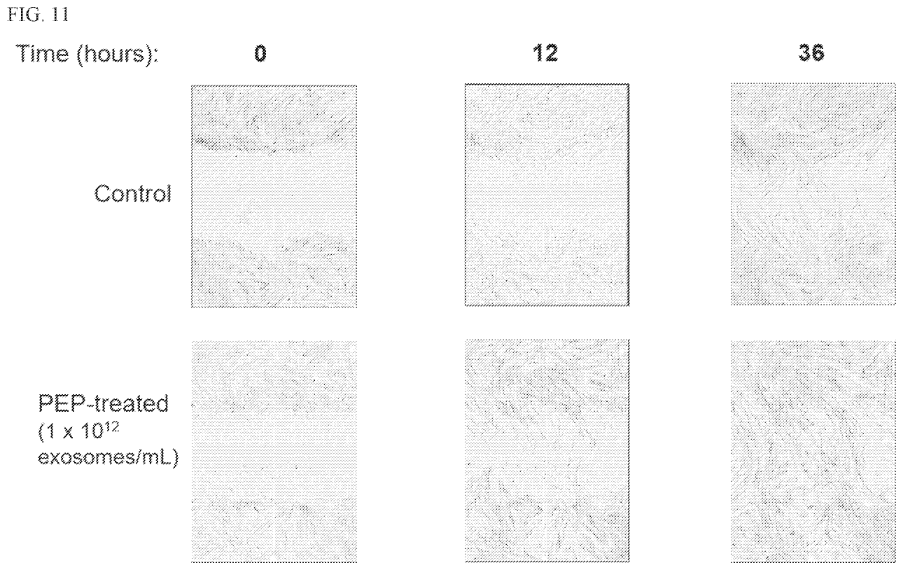

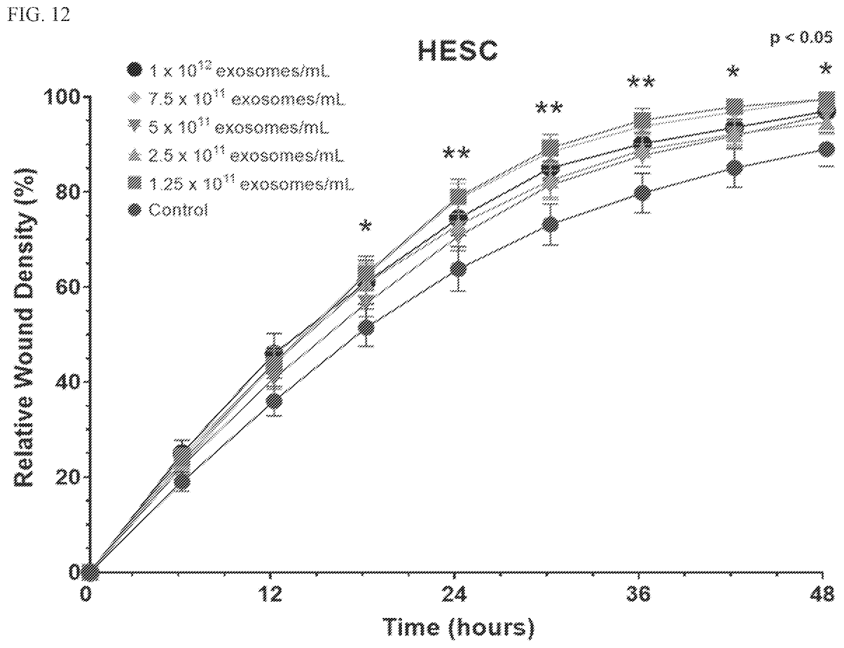

Similar results were observed in the scratch assay using HESCs. FIG. 11 shows a representative image of wound closure in HESCs at 12 hours and 36 hours after treatment. The wound closure in the PEP-treated HESCs was noticeably greater than the untreated HESCs. While the difference in wound healing between PEP-treated and untreated HESCs was not as great as the difference observed in HEC-1A cells, all concentrations of PEP induced greater wound healing than the untreated HESCs (FIG. 12).

This disclosure therefore describes PEP compositions and methods for treating endometrial tissue in a subject. In some cases, the endometrial tissue may be damaged. Damage to endometrial can include scar tissue in the endometrium and/or adhesions in the endometrium. In some cases, the subject may have, or be at risk for developing, Asherman's Syndrome, which is characterized by scar tissue and/or adhesions in the uterus or cervix.

Treating a condition can be prophylactic or, alternatively, can be initiated after the subject exhibits one or more symptoms or clinical signs of the condition. Treatment that is prophylactic—e.g., initiated before a subject manifests a symptom or clinical sign of the condition—is referred to herein as treatment of a subject that is “at risk” of having the condition. As used herein, the term “at risk” refers to a subject that may or may not actually possess the described risk. Thus, for example, a subject “at risk” of having damaged endometrial tissue is a subject possessing one or more risk factors associated with the condition such as, for example, genetic predisposition, ancestry, age, or medical history (e.g., dilation and curettage (D&C), certain pregnancy complications, scheduled uterine surgery, etc.). Treatment may also be continued after symptoms have resolved, for example to prevent or delay their recurrence.

Accordingly, a PEP composition can be administered before, during, or after the subject first exhibits a symptom or clinical sign of damaged endometrial tissue. Treatment initiated before the subject first exhibits a symptom or clinical sign associated with damaged endometrial tissue may result in decreasing the likelihood that the subject experiences clinical evidence of damaged endometrial tissue compared to a subject to which the composition is not administered, decreasing the severity of symptoms and/or clinical signs of damaged endometrial tissue, and/or completely resolving the damaged endometrial tissue. Treatment initiated after the subject first exhibits a symptom or clinical sign associated with damaged endometrial tissue may result in decreasing the severity of symptoms and/or clinical signs of damaged endometrial tissue compared to a subject to which the composition is not administered, and/or completely resolving the damaged endometrial tissue.

In one or more embodiments, the PEP composition can be administered preparationally—in preparation of—a subsequent event. For example, the PEP composition may be administered to prepare the endometrium to be more receptive to implantation of a fertilized egg. The egg may be fertilized in vitro (e.g., a product of an in vitro fertilization (IVF) procedure) or in vivo (e.g., a product of artificial insemination or natural fertilization).

Thus, in one or more embodiments, the method includes administering an effective amount of the PEP composition to a subject having, or at risk of having, damaged endometrial tissue. In this context, an “effective amount” is an amount effective to reduce, limit progression, ameliorate, or resolve, to any extent, a symptom or clinical sign related to the condition. Exemplary symptoms or clinical signs of damaged endometrial tissue include, but are not limited to, light menstrual periods (hypomenorrhea), absence of menstrual periods (amenorrhea), severe menstrual cramping, difficulty becoming pregnant, or difficulty maintaining pregnancy.

In other embodiments, the method includes administering an effective amount of the PEP composition to a subject in need of preparational treatment of the endometrium. In this context, an “effective amount” is an amount effective to prepare the endometrium to be more receptive to implantation of a fertilized egg that the endometrium of a similar subject not receiving the preparational treatment.

Generally, the compositions include PEP and a pharmaceutically acceptable carrier. In a surgical setting, the PEP may be combined with a carrier that is suitable for delivering the PEP preparation to endometrial tissue such as, for example, a surgical glue, a tissue adhesive, and/or a supportive matrix (e.g., a collagen scaffold).

Thus, the method includes administering an effective amount of the PEP preparation to the subject and, in some cases, directly to endometrial tissue. In this context, an “effective amount” is an amount effective to promote endometrial cell proliferation and/or promote wound closure in endometrial tissue.

As used herein, a “subject” can be a human or any non-human animal. Exemplary non-human animal subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

PEP may be formulated with a pharmaceutically acceptable carrier to form a pharmaceutical composition. As used herein, “carrier” includes any solvent, dispersion medium, vehicle, hydrogel, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue or tissue adhesive.

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., application to nervous tissue exposed during surgery, intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered. Thus, in one or more embodiments, the method can include administering sufficient PEP to provide a dose of, for example, from about 1×10⁶ PEP exosomes to about 1×10¹⁵ PEP exosomes to the subject, although in some embodiments the methods may be performed by administering PEP in a dose outside this range.

In one or more embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10¹⁰ PEP exosomes, at least 1×10¹¹ PEP exosomes, at least 1.25×10¹¹ PEP exosomes, at least 2×10¹¹ PEP exosomes, at least 2.5×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 7.5×10¹¹ PEP exosomes, at least 8×10¹¹ PEP exosomes, at least 9×10¹¹ PEP exosomes, at least 1×10¹² PEP exosomes, at least 2×10¹² PEP exosomes, at least 3×10¹² PEP exosomes, at least 4×10¹² PEP exosomes, at least 5×10¹² PEP exosomes, at least 1×10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In one or more embodiments, the method can include administering sufficient PEP to provide a dose characterized by a range having endpoints defined by any minimum dose identified above and any maximum dose that is greater than the minimum dose. For example, in one or more embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, a dose of from 5×10¹² to 1×10¹³ PEP exosomes, or a dose of 1.25×10¹¹ PEP exosomes to 1×10¹² PEP exosomes. In certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 1.25×10¹¹ PEP exosomes, 2.5×10¹¹ PEP exosomes 5×10¹¹ PEP exosomes, 7.5×10¹¹ PEP exosomes, 1×10¹² PEP exosomes, 5×10¹² PEP exosomes, 1×10¹³ PEP exosomes, or 1×10¹⁴ PEP exosomes.

Alternatively, in one or more embodiments, the method can include administering sufficient PEP to provide a dose of, for example, from about a 0.01% solution to a 100% solution to the subject, although in some embodiments the methods may be performed by administering PEP in a dose outside this range. As used herein, a 100% solution of PEP refers to approximately 75 mg of PEP solubilized in 1 ml of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell-conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any minimum dose identified above and any maximum dose that is greater than the minimum dose. For example, in one or more embodiments, the method can include administering sufficient PEP to provide a dose of from 1% to 50% such as, for example, a dose of from 5% to 20%. In certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered all at once, continuously for a prescribed period of time, or in multiple discrete administrations. When multiple administrations are used, the amount of each administration may be the same or different. For example, a prescribed daily dose may be administered as a single dose, continuously over 24 hours, or as two administrations, which may be equal or unequal. When multiple administrations are used to deliver a single dose, the interval between administrations may be the same or different. In certain embodiments, PEP may be administered from a one-time administration, for example, during a surgical procedure.

In certain embodiments in which multiple administrations of the PEP composition are administered to the subject, the PEP composition may be administered as needed to treat damaged endometrial tissue to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP at an interval (for two administrations) or intervals (for more than two administrations) characterized by a range having endpoints defined by any minimum interval identified above and any maximum interval that is greater than the minimum interval. For example, in one or more embodiments, the method can include multiple administrations of PEP at an interval or intervals of from one day to six months such as, for example, from three days to ten days. In certain embodiments, the method can include multiple administrations of PEP at an interval that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique profile—e.g., protein composition and/or gene expression. In this way, the PEP composition can provide a broader spectrum of endometrial tissue healing activity than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### Exemplary Embodiments

Embodiment 1 is a method for treating endometrial tissue in a subject, the method comprising administering to the subject an amount of a PEP preparation effective to treat the subject's endometrial tissue.

Embodiment 2 is the method of Embodiment 1, wherein the subject has damaged endometrial tissue.

Embodiment 3 is the method of any one of Embodiments 1 or 2, wherein the subject has Asherman's Syndrome.

Embodiment 4 is the method of any one of Embodiments 1-3, wherein the subject has been subjected to uterine surgery.

Embodiment 5 is the method of any one of Embodiments 1-4, wherein the subject is at risk of having damaged endometrial tissue.

Embodiment 6 is the method of any one of Embodiments 1-5, wherein the PEP preparation is administered to the subject prior to uterine surgery.

Embodiment 7 is the method of any one of Embodiments 1-6, wherein the PEP preparation is administered in preparation for a procedure.

Embodiment 8 is the method of any one of Embodiments 1-7, wherein the PEP preparation is administered prior to an in vitro fertilization procedure.

Embodiment 9 is a method of promoting proliferation of endometrial cells, the method comprising contacting the endometrial cells with an amount of a PEP preparation effective to promote proliferation of the endometrial cells.

Embodiment 10 is a method of promoting wound closure by endometrial cells, the method comprising administering to endometrial cells into which a wound has been introduced an amount of a PEP preparation effective to promote closure of the wound.

Embodiment 11 is the method of any one of Embodiments 1-10, wherein the PEP preparation comprises CD63⁺ exosomes.

Embodiment 12 is the method of any one of Embodiments 1-11, wherein the PEP preparation has a concentration of at least 1×10⁹ PEP exosomes/mL.

Embodiment 13 is the method of any one of Embodiments 1-11, wherein the PEP preparation has a concentration of 1×10⁹ to 1×10¹¹ PEP exosomes/mL.

Embodiment 14 is the method of any one of Embodiments 10 to 13, wherein the wound closes twice as fast as a comparable wound not treated with the PEP preparation.

Embodiment 15 is the method of any one of Embodiments 9 or 11-13, wherein the cells proliferate at least twice as quickly as comparable cells not treated with the PEP preparation. Embodiment 16 is the method of any one of Embodiments 1-8, wherein the treatment prevents formation of adhesions.

Embodiment 17 is the method of any one of Embodiments 1-8, wherein the treatment restores functionality of the uterus.

### Examples

### DiR Labeling of PEP

Cryodesiccated PEP was prepared as previously described (International Publication No. WO 2019/118817), then reconstituted in heparinized serum-free media and incubated with a lipophilic dye (DiR) to label the exosomes. This preparation was then filtered, washed using phosphate buffered saline (PBS) and administered to cells in culture. Approximately 30,000 HEC-1A cells or HESCs were plated into each side of a dual chamber glass slide. PEP was reconstituted with 4.6 mL of serum-free media plus 400 μl of heparin. Exosomes were labeled with the lipophilic, membrane-specific, far-red dye DiR (1,1′-dioctadecyl-3,3,3′,3′-tetramethylindotricarbocyanide iodide). 1 mL of the reconstituted PEP was added to one side of the dual chamber slide. 1 mL of serum free media without PEP was added to the other side, and the slide was incubated for one hour, then rinsed and fixed with paraformaldehyde (PFA) overnight

After fixing with PFA, the cells were stained using an anti-CD63 antibody, phalloidin, and DAPI. The anti-CD63 antibody was visualized using a secondary antibody labeled with ALEXA FLUOR 555. Phalloidin was used to visualize actin. DAPI was used to visualize nuclei. A coverslip was mounted with prolonged gold anti-fade reagent with DAPI. Images of each label were obtained via confocal fluorescence microscopy, shown in FIG. 1 and FIG. 3.

Both HESC and HEC-1A cells that had been treated with PEP showed punctate cytosolic localization of DiR, indicating that the PEP had been taken up by the cells and localized to a distinct perinuclear location (FIG. 1, FIG. 3). This observation aligns with knowledge that endocytic vesicles, including PEP, are recognized and localized by cells upon endocytosis. HEC-1A cells not treated with PEP show low levels of CD63 staining, which aligns with the knowledge that HEC-1A inherently has exosomes, which are stained by CD63. HESC showed minimal levels of CD63 staining.

The image data of FIG. 1 and FIG. 3 is quantified in FIG. 2 and FIG. 4. A statistically significant difference in the amount of DiR and CD63 between treated and untreated cells was observed in both HEC-1A cells (FIG. 2) and HESCs (FIG. 4).

### Cell Proliferation Assay

To test cell proliferation, the INCUCYTE S3 imaging system (Sartorius AG, Göttingen, Germany) was used to image and count cells throughout treatment with PEP exosomes.

To perform the assay, cells were thawed from cryopreservation and subsequently plated into a 96-well tissue culture plate in standard growth media. HEC-1A cells were plated at approximately 5,000 cells/well; HESCs were plated at approximately 1,000 cells/well. The plated cells were rinsed one with PBS. 50 μl of serum free media was added to the control wells. Experimental wells were treated with 50 μl of 1.25×10¹¹ PEP exosomes/ml, 50 μl of 2.5×10¹¹ PEP exosomes/ml, 50 μl of 5×10¹¹ PEP exosomes/ml, 50 μl of 7.5×10¹¹ PEP exosomes/ml, or 50 μl of 1×10¹² PEP exosomes/ml. The plate was then incubated in a live-cell analytical system for 48 hours (INCUCYTE, Sartorius AG, Göttingen, Germany).

An imaging system (INCUCYTE C3, Sartorius AG, Göttingen, Germany) acquired images of the cells within each well at regular time intervals throughout the incubation period. Upon completion of the incubation period, images were analyzed using the accompanying software package (INCUCYTE, Sartorius AG, Göttingen, Germany) to assess cell confluency in each well.

Representative images showing control untreated HEC-1A cells and HEC-1A cells treated with 50 μl of 1×10¹² exosomes/mL (5×10¹⁰ total exosomes) are shown in FIG. 5. HEC-1A cells treated with PEP exosomes proliferated more quickly than control cells. FIG. 6 shows quantified data from multiple images of HEC-1A cells treated with each concentration of PEP exosomes. Cells treated with higher concentrations of PEP proliferated more quickly.

Representative images showing control untreated HESCs and HESCs treated with 50 μl of 1×10¹² exosomes/mL (5×10¹⁰ total exosomes) are shown in FIG. 7. HESCs treated with PEP exosomes proliferated more quickly than control cells, similarly to HEC-1A cells. FIG. 8 shows quantified data from multiple images of HESCs treated with each concentration of PEP exosomes. Surprisingly, HESCs treated with the highest concentration of PEP exosomes showed nearly 400% proliferation compared to control cells, which showed less than 50% proliferation over 48 hours. HESCs treated with the three highest concentrations of PEP showed statistically significantly increased levels of proliferation only six hours after treatment.

### Scratch Assay (In Vitro Wound Healing)

Cells were plated in a 96-well plate using a multichannel pipette and allowed to attach and proliferate to confluency. HEC-1A cells were plated at a confluency 50,000 cells/well. HESCs were plated at a confluency of 30,000 cells/well.

When the cells were confluent, a wound maker tool (Sartorius AG, Göttingen, Germany) was used to create homogeneous scratch wounds, disrupting the monolayer, in the center of each well. After a uniform scratch was confirmed via microscopy, the wells were rinsed once with PBS and either serum-free media or media with different concentrations of PEP was added as described for the cell proliferation assay. The plate was then placed in a live-cell analytical system (INCUCYTE, Sartorius AG, Göttingen, Germany) housed within a tissue culture incubator for 48 hours.

Images of each well were acquired and analyzed as described above for the cell proliferation assay.

Representative images showing wound healing of control untreated HEC-1A cells and HEC-1A cells treated with 50 μl of 1×10¹² exosomes/mL (5×10¹⁰ total exosomes) are shown in FIG. 9. After 36 hours, HEC-1A cells treated with PEP exosomes had grown to nearly cover the wound. In contrast, untreated HEC-1A cells had only partially grown to cover the wound at 36 hours. FIG. 10 shows quantification of image data for HEC-1A cells treated with each concentration of PEP exosomes. Cells treated with PEP exosomes showed more wound healing as measured by wound density. This effect was generally dose-dependent. Six hours after treatment, cells treated with 50 μl of 1×10¹² exosomes/mL and 50 μl of 5×10¹¹ exosomes/mL had statistically significant wound density as compared to untreated HEC-1A cells. 18 hours after treatment, cells treated with each concentration of PEP exosomes had statistically significant wound density as compared to untreated HEC-1A cells.

Representative images showing wound healing of control untreated HESCs and HESCs treated with 50 μl of 1×10¹² exosomes/mL (5×10¹⁰ total exosomes) are shown in FIG. 11. After 36 hours, HESCs treated with PEP exosomes had grown to cover the wound. After 36 hours, control untreated HESCs had grown to partially, but not completely cover the wound. FIG. 12 shows quantification of image data for HESCs treated with each concentration of PEP exosomes. Interestingly, the effect did not appear to be dose-dependent, rather, cells treated with any concentration of PEP exosomes showed increased wound density compared to untreated HESCs. After 36 hours, HESCs treated with 50 μl of 1×10¹² exosomes/mL, 50 μl of 7.5×10¹¹ exosomes/mL, or 50 μl of 1.25×10¹¹ exosomes/mL showed statistically significantly higher wound density than control HESCs. These results suggest an unexpected relationship between administered PEP exosome dose and HESC wound healing.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for treating endometrial tissue in a subject, the method comprising administering to the subject an amount of a PEP preparation effective to treat the subject's endometrial tissue.

2. The method of claim 1, wherein the subject has damaged endometrial tissue.

3. The method of claim 1, wherein the subject has Asherman's Syndrome.

4. The method of claim 1, wherein the subject has been subjected to uterine surgery.

5. The method of claim 1, wherein the subject is at risk of having damaged endometrial tissue.

6. The method of claim 5, wherein the PEP preparation is administered to the subject prior to uterine surgery.

7. The method of claim 1, wherein the PEP preparation is administered in preparation for a procedure.

8. The method of claim 7, wherein the PEP preparation is administered prior to an in vitro fertilization procedure.

9. A method of promoting proliferation of endometrial cells, the method comprising contacting the endometrial cells with an amount of a PEP preparation effective to promote proliferation of the endometrial cells.

10. A method of promoting wound closure by endometrial cells, the method comprising administering to endometrial cells into which a wound has been introduced an amount of a PEP preparation effective to promote closure of the wound.

11. The method of claim 9, wherein the subject has damaged endometrial tissue.

12. The method of claim 9, wherein the subject has Asherman's Syndrome.

13. The method of claim 9, wherein the subject has been subjected to uterine surgery.

14. The method of claim 9, wherein the subject is at risk of having damaged endometrial tissue.

15. The method of claim 14, wherein the PEP preparation is administered to the subject prior to uterine surgery.

16. The method of claim 9, wherein the PEP preparation is administered in preparation for a procedure.

17. The method of claim 16, wherein the PEP preparation is administered prior to an in vitro fertilization procedure.

18. The method of claim 10, wherein the subject has damaged endometrial tissue.

19. The method of claim 10, wherein the subject has Asherman's Syndrome.

20. The method of claim 10, wherein the subject has been subjected to uterine surgery.

21. The method of claim 10, wherein the subject is at risk of having damaged endometrial tissue.

22. The method of claim 21, wherein the PEP preparation is administered to the subject prior to uterine surgery.

23. The method of claim 10, wherein the PEP preparation is administered in preparation for a procedure.

24. The method of claim 23, wherein the PEP preparation is administered prior to an in vitro fertilization procedure.
